(19) United States
(12) Patent Application Publication
(10) Pub. No.: US 2020/0219592 A1
(43) Pub. Date: Jul. 9, 2020

(54) ESTIMATING PATIENT ADHERENCE TO PRESCRIBED THERAPY

(71) Applicant: International Business Machines Corporation, Armonk, NY (US)

(72) Inventors: Amit Gruber, Tel Aviv (IL); Chen Yanover, Zichron Yaakov (IL); Yishai Shimoni, Givatayim (IL)

(21) Appl. No.: 16/240,815

(22) Filed: Jan. 7, 2019

(51) Int. Cl. G16H 10/20; G16H 50/50

(52) U.S. Cl. CPC G16H 10/20; G16H 50/30; G16H 50/50

(57) ABSTRACT

A method comprising receiving a dataset comprising: (i) a treatment plan for a subject, the treatment plan comprising a plurality of treatment events scheduled at specified intervals, and (ii) clinical outcomes of said subjects observed during said treatment plan; and automatically analyzing said dataset to determine adherence by said subject to said treatment plan.

### BACKGROUND

The invention relates to the field of automated data analysis.

Studies have shown that patients only partially follow prescribed therapy, and that long-term drug regimens are not always properly observed. Partial or complete non-adherence by a patient may affect therapy outcome and future treatment choices. Due to the prevalence of patient non-adherence (studies have shown that in some cases, patient adherence can be as low as 50%-60%), and the importance of estimating patient adherence, this problem receives much attention. Because physicians oftentimes are unaware of the adherence level of a patient, some uncertainty arises regarding the effectiveness of the prescribed therapy. For example, when a therapy fails, it may be due to a drug being ineffective (which calls for treatment modification), but it also may be due to non-adherence (in which case drug effectiveness may not be assessed). This may affect caregivers treatment selection, as well as research and analysis of treatment effectiveness.

The foregoing examples of the related art and limitations related therewith are intended to be illustrative and not exclusive. Other limitations of the related art will become apparent to those of skill in the art upon a reading of the specification and a study of the figures.

### SUMMARY

The following embodiments and aspects thereof are described and illustrated in conjunction with systems, tools and methods which are meant to be exemplary and illustrative, not limiting in scope.

There is provided, in an embodiment, a system comprising at least one hardware processor; and a non-transitory computer-readable storage medium having stored thereon program instructions, the program instructions executable by the at least one hardware processor to: receive a dataset comprising: (i) a treatment plan for a subject, the treatment plan comprising a plurality of treatment events scheduled at specified intervals, and (ii) clinical outcomes of said subjects observed during said treatment plan, and automatically analyze said dataset to determine adherence by said subject to said treatment plan.

There is also provided, in an embodiment, a method comprising receiving a dataset comprising: (i) a treatment plan for a subject, the treatment plan comprising a plurality of treatment events scheduled at specified intervals, and (ii) clinical outcomes of said subjects observed during said treatment plan; and automatically analyzing said dataset to determine adherence by said subject to said treatment plan.

There is further provided, in an embodiment, a computer program product comprising a non-transitory computer-readable storage medium having program code embodied therewith, the program code executable by at least one hardware processor to: receive a dataset comprising: (i) a treatment plan for a subject, the treatment plan comprising a plurality of treatment events scheduled at specified intervals, and (ii) clinical outcomes of said subjects observed during said treatment plan; and automatically analyze said dataset to determine adherence by said subject to said treatment plan.

In some embodiments, the analyzing comprises applying one or more stochastic models to said dataset.

In some embodiments, the one or more models includes a Factorial Hidden Markov Model.

In some embodiments, said analyzing is performed, at least in part, via one or more approximate learning methods.

In some embodiments, the one or more approximate learning methods include Collapsed Gibbs Sampling.

In some embodiments, said analyzing further determines effectiveness of said treatment plan.

In addition to the exemplary aspects and embodiments described above, further aspects and embodiments will become apparent by reference to the figures and by study of the following detailed description.

### BRIEF DESCRIPTION OF THE FIGURES

Exemplary embodiments are illustrated in referenced figures. Dimensions of components and features shown in the figures are generally chosen for convenience and clarity of presentation and are not necessarily shown to scale. The figures are listed below.

FIG. 1 illustrates exemplary treatment outcome clinical data;

FIG. 2 is a block diagram of an exemplary system for automated estimation patient adherence to a prescribed treatment regimen, according to an embodiment;

FIG. 3 is a flowchart illustrating the functional steps of a process automatically estimating patient adherence to a prescribed treatment regimen, according to an embodiment; and

FIG. 4 schematically illustrates an exemplary Factorial HMM model, according to an embodiment.

### DETAILED DESCRIPTION

Disclosed herein are a system, method, and computer program product for automatically estimating patient adherence to a prescribed treatment regimen based, at least in part, on analysis of patient clinical data. In some embodiments, the present invention may provide decision support for effective long-term drug therapy.

There are several types of treatment non-adherence. For example, ‘primary’ non-adherence occurs when a healthcare provider writes prescription, but the medication is never filled or initiated. A second example may be non-persistence, when a patient decides to stop taking a medication after starting it, without being advised by a healthcare professional to do so. Another example may be a non-conforming patient, who may skip doses, take medications at incorrect times or at incorrect doses, or even take more than the prescribed dosage. The consequences of non-adherence are waste of medication, disease progression, reduced functional abilities, a lower quality of life, and increased use of medical resources such as nursing homes, hospital visits and hospital admissions (see, for example, Col N. et al., “The role of medication noncompliance and adverse drug reactions in hospitalizations of the elderly”, Arch Intern Med 1990 April 150(4):841-845; and Sullivan S. et al., “Noncompliance with medication regimens and subsequent hospitalizations: A literature analysis and cost of hospitalization estimate”, J Res Pharmaceut. Econ. 1990; 2:19-33.1).

Various methods have been proposed and are in use to measure adherence. Direct methods include directly-observed therapy, measurement of the level of a drug or its metabolite in blood or urine, and detection or measurement of a biological marker added to the drug formulation in the blood. Direct approaches are one of the most accurate methods of measuring adherence, however, they are costly and time- and resource-consuming. Self-reporting methods include patient questionnaires, patient self-reports, as well as patient diaries, which may be inconsistent and unreliable.

Accordingly, in some embodiments, the present invention provides for an automated algorithm which analyzes patient data including (i) a prescribed treatment regimen (e.g., one or more courses of medications), and (ii) a time series of clinical variables (e.g., viral load in the patient). In some embodiments, this analysis is based on the notion that therapy typically begins to affect the clinical variables of a patient within a specified period of time after taking the medication, and the effect of the medication typically may be expected to last for another specified period of time.

In some embodiments, based on this automated analysis, an algorithm of the present invention may be configured for determining a level of patient adherence to the treatment regimen. In some embodiments, based on this analysis, an algorithm of the present invention may further be configured for determining a state or level of clinical variables related to patient non-adherence, such as viral load, which may indicate a pathogen developing drug resistance.

In some embodiments, the present invention may be configured for distinguishing between cases of therapy failure due to drug ineffectiveness (e.g., due to a pathogen developing resistance to the drug), and inconsistent therapy results, which may indicate partial adherence or non-adherence by the patient.

In some embodiments, the present invention may be applied in cases where patient clinical variables and/or other physical outcomes are regularly observed and recorded, e.g., in the case of chronic diseases (such as HIV), or conditions (such as epilepsy, diabetes, etc.).

A potential advantage of the present invention is, therefore, in that it allows for an automated algorithm for identifying cases of non-adherence in patients, based solely on observed and/or recorded medical history, without requiring repeated costly medical tests and/or examinations, and without having to rely on unreliable patient self-reporting.

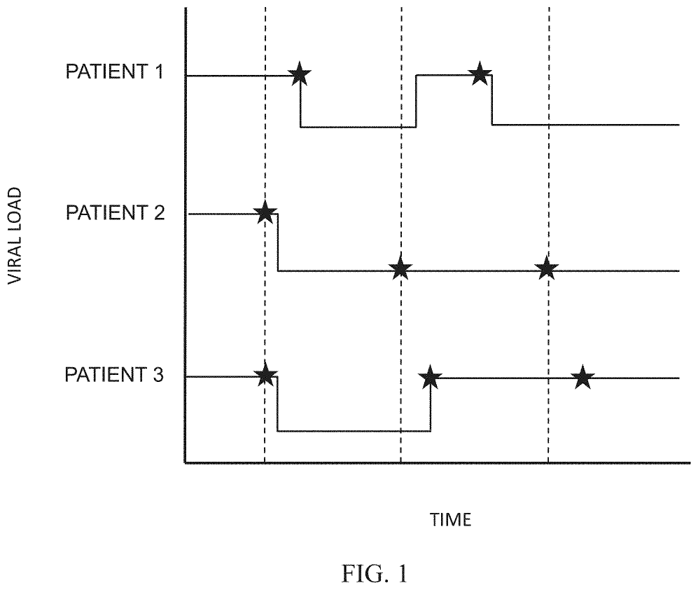

FIG. 1 illustrates exemplary clinical data time series for three patients, where the graphs indicate viral load levels in each patient, the dashed vertical lines indicate prescribed treatment intervals, and the stars indicate actual treatment events (e.g., the patient actually taking the prescribed medication). As can be seen, patient 1 has not adhered to the prescribed medication regimen. This may be inferred, e.g., from the variations in the viral load levels and the fact that measured decreases in viral load levels are not temporally aligned with the prescribed medication intervals. At the same time, when patient 1 does take medication, viral load levels show a decrease, indicating a smaller likelihood that drug resistance has been built up by the pathogen. Patient 2 has been adhering to the medication regimen, as indicated by the sustained decrease in viral load levels, which also indicates lack of resistance build up. Patient 3 has not been adhering to the medication regimen, which may be indicated by inconsistent viral load level results. At the same time, a persistent return of viral load to initial levels may also indicate virus resistance build up.

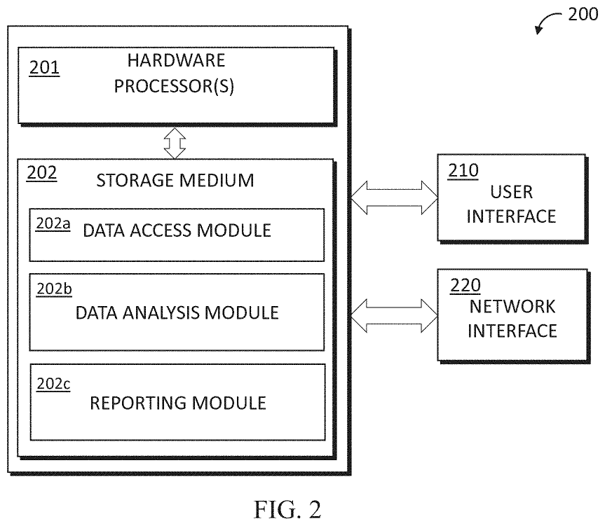

FIG. 2 is a block diagram of an exemplary system 200 for automated estimation of patient adherence to a prescribed treatment regimen, according to an embodiment. System 200 may comprise one or more hardware processors 201, a non-transitory computer-readable storage medium 202, a user interface 210, and a network interface 220. System 200 as described herein is only an exemplary embodiment of the present invention, and in practice may be implemented in hardware, software only, or a combination of both hardware and software. In some embodiments, system 200 may be any computing platform capable of performing the described functions. System 200 may have more or fewer components and modules than shown, may combine two or more of the components, or may have a different configuration or arrangement of the components. In various embodiments, system 200 may comprise one or more dedicated hardware devices, one or more software modules, and/or may form an addition to or extension to an existing device.

Storage medium 202 may have encoded thereon software instructions or components configured to operate a processing unit (also “hardware processor,” “CPU,” or simply “processor”), such as hardware processor(s) 201. In some embodiments, the software components may include an operating system, including various software components and/or drivers for controlling and managing general system tasks (e.g., memory management, storage device control, power management, etc.), and facilitating communication between various hardware and software components. In some embodiments, the software instructions may be segmented into functional modules, such as data access module 202a, data analyzer 202b, and/or reporting module 202c.

In some embodiments, data analyzer 202b may employ one or more computerized data analysis models. In some embodiments, such models may be configured for inferring a probability that a failure in prescribed therapy is due to the development of drug resistance and/or failure to adhere to the prescribed regimen. In some embodiments, a Factorial Hidden Markov Model (HMM) consisting of one or more chains interconnected through observation may be employed (see, e.g., Z Ghahramani, M. I. Jordan, and P. Smyth. Factorial hidden markov models. Machine learning, 29(2-3): 245-273, 1997.)

An exemplary HMM model was described by the present inventors in Gruber [2018], which is incorporated herein by reference (see, Gruber A., et al., “Factorial HMMs with Collapsed Gibbs Sampling for Optimizing Long-term HIV Therapy”, Proceedings of the 21st International Conference on Artificial Intelligence and Statistics (AISTATS) 2018, Lanzarote, Spain. PMLR: Volume 84).

While the example embodiments described further below uses a Factorial HMM, it will be appreciated that other embodiments may implement alternative models. Moreover, some models may be applicable to specific treatment regimens, e.g., those involving bacterial or viral infections, while other models may be exclusive to other types of treatments.

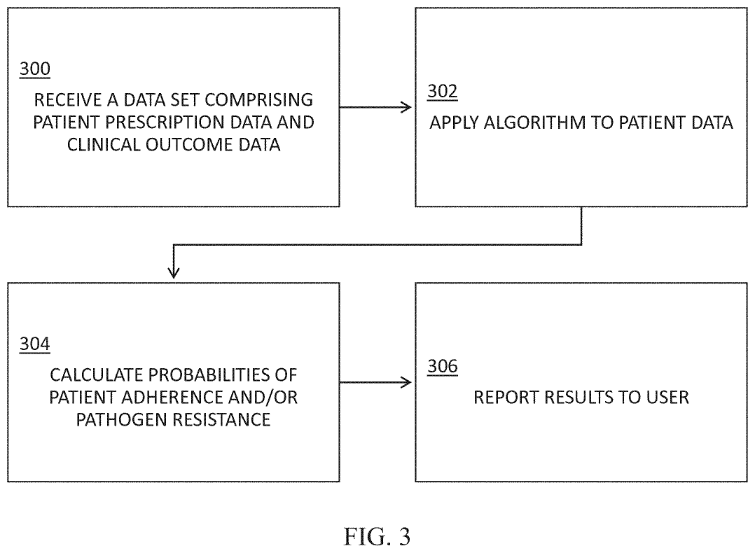

An overview of the functional steps in a process for automated estimating of patient adherence to a prescribed treatment regimen will now be provided with continued reference to FIG. 2 and the flowchart in FIG. 3.

In some embodiments, at a step 300 in FIG. 3, system 200 may receive as input a data set comprising (i) prescribed treatment intervals relating to one or more treatment regimens, and (ii) one or more time series detailing patient clinical outcomes associated with such treatment regimens.

In some embodiments, system 200 may be configured for accessing such patient data through data access module 202a. For example, data access module 202a may access a patient-specific medication regimen and/or other treatment regimen from a healthcare provider, which may include indicated prescribed treatment intervals for the patient.

In some embodiments, data access module 202a receives patient data remotely from user of system 200, e.g., via user interface 210 and network interface 220. In other embodiments, data access module 202a may retrieve patient data on a recurring basis or upon triggers such as uploading of new data or detection of an appointment with a particular patient. Patient data may include test results, current and previous treatments, outcomes to current and previous treatments, patient demographic data, patient activity data, and the like.

For example, in the case of an HIV patient, a treatment regimen may prescribe combined antiretroviral therapy (cART) pills to be taken at specified intervals (e.g., daily) on a continuing basis. In addition, data access module 202a may access a time series of reports indicating one or more therapy outcomes. Such reports may include physical measurements and/or clinical variables of the patient obtained, e.g., during the course of the treatment regimen. For example, data access module 202a may access electronic health records (EHR) relating to the patient, which include the patient's clinical measurements. Thus, in the case of the HIV patient, EHR data may indicate historical HIV viral load levels as measured in the patient over a period of time.

At a step 302, data analysis module 202b may be configured for analyzing the accessed reports. For example, data analysis module 202b may temporally correlate patient therapy outcomes data with prescribed treatment events, to infer one or more adherence-related parameters with respect to the patient. Data analysis module 202b may then apply an algorithm of the present invention to derive probabilities indicating whether a therapy failure is the result of patient partial or complete non-adherence, and/or drug resistance buildup. For example, when a patient does not strictly adhere to a prescribed treatment regimen (e.g., by taking medication at different times than prescribed, or missing one or more treatment events altogether), patient therapy outcomes may fail to show progression and/or a sustained improvement in clinical outcomes (e.g., viral load), and/or show variability of outcomes which is not temporally-aligned with the prescribed medication intervals. This may indicate a greater probability of patient non-adherence, with a smaller probability of drug resistance buildup. Conversely, patient clinical outcomes that are aligned with the prescribed treatment regimen (e.g., a sustained decrease in viral load levels as the treatment regimen progresses), may indicate a higher probability of patient adherence with a smaller probability of drug resistance buildup. In a third example, patient clinical outcomes which show a lack of progression or even regression in clinical outcomes over time may indicate a higher probability of drug resistance build up.

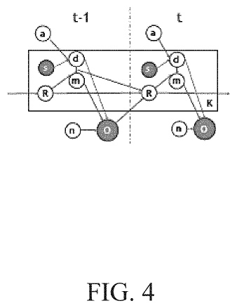

FIG. 4 schematically illustrates a Factorial HMM model which may be employed by the present invention, as described by Gruber [2018]. The Factorial HMM consists of multiple chains interconnected through observation, wherein each chain corresponds to a possible resistance with respect to a specific drug and its evolution over time while the observation connecting the chains is the therapy outcome. More specifically, in this embodiment, pathogen sensitivity may be modeled to each available drug as a Markov chain with a hidden state comprising two variables: a binary variable (R in FIG. 4) indicating whether a perpetual resistance to that drug has been acquired, and a binary variable (m in FIG. 4) indicating the instantaneous existence of a drug resistant mutation.

In some embodiments, the generative process of the Factorial HMM model may further be configured for estimating patient adherence to prescribed drug therapy. For example, let a_{i,t} be a binary variable denoting whether patient i has taken the prescribed drugs at time t, and let pᵢ^{A}=Pr(a_{i,t}=1) be the patient-specific adherence probability. In the case of cART therapy, typically all therapy drugs are contained in a single pill, such that all drugs can either be taken or not at a specific time t, and therefore a single adherence a_{i,t} variable is sufficient for all drugs. In other implementations, more than one adherence variable may be employed.

The generative process may then he described as follows:

1. For all patients i=1, . . . , N
(a) draw pᵢ^{A}˜Beta (α)
(b) for t=1, . . . , T
   i. draw a_{i,t}˜pᵢ^{A}
   ii. set d_{i,t,k}=a_{i,t}∧s_{i,t,k} for all k=1, . . . , K,
      where d_{i,t,k} is a binary variable indicating whether drug k was prescribed to patient i under the therapy at time t, N is the number of patients, T is the number of recorded treatments and outcomes, K is the number of drug compounds available for treating the disease, Beta is the Beta distribution, and α is a Beta hyper-parameter. These steps set d_{i,t,k} to the drugs taken in practice by patient i at time t which are unobserved. Once these drugs have been set, the generative process proceeds as described below.

In some embodiments, the generative process of the Factorial HMM model may be configured for drug resistance in a patient, wherein the following focuses on a single patient and omits the patient-specific index. For example, given d (as defined above), let: Oₜ be the multi-drug treatment outcome (Oₜ=1 for a successful outcome), m_{t,k} be a binary variable representing the existence of mutations resistant to drug k in the blood at time t, and R_{t,k} be a binary variable representing whether permanent resistance to drug k already exists at time t. If permanent resistance was already acquired at time t (i.e., R_{t,k}=1), then the drug resistant pathogen may also be found in the present case m_{t,k}=1. Otherwise, when R_{t,k}=0, when drug k is taken (d_{t,k}=1), new drug resistant mutations may appear with some probability which may be denoted as pₖ^{M}. If the drug is not taken at time t, no such mutations will prevail. The following equation summarizes this relation:

$$P r ( {m}_{t , k} = 1 \vert {R}_{t , k} , {d}_{t , k} ) = \{ \begin{matrix}1 , if {R}_{t , k} = 1 \\ {p}_{k}^{M} , if {R}_{t , k} = 0 , {d}_{t , k} = 1 . \\ 0 , otherwsie\end{matrix} \qquad ( 1 )$$

When drug resistant mutations appear, (mₜ=1), and are not suppressed (Oₜ=0), these may form reservoirs, and the virus will consequently acquire permanent resistance to drug k (R_{t+1,k}=1). The probability for this event may be denoted by p^{C}. There are two conditions for this situation: (i) mutations resistant to drug k have emerged, and (ii) the treatment fails, i.e., no other drug taken at time t is successful in suppressing the virus. It is also possible, that viral reservoirs resistant to drug k had already existed at time t and will be preserved at time t+1. Finally, there is also a low probability for a new infection by a strain resistant to drug k between time t and t+1, leading to the creation of a new viral reservoir. The probability for this event may be denoted by p^{I}. In practice, p^{C}=1−ϵ is assumed, and p^{I}=ϵ for a small fixed ϵ.

$$Pr ( {R}_{t + 1 , k} = 1 \vert {R}_{t , k} , {O}_{t , k} , {O}_{t} ) = \{ \begin{matrix}1 - ϵ , if {R}_{t , k} = 1 \\ 1 - ϵ , if {R}_{t , k} = 0 , {m}_{t , k} = 1 , {O}_{t} = 0 . \\ ϵ , otherwsie\end{matrix} \qquad ( 2 )$$

A prior Pₖ^{R}^{₀}=Pr(R_{1,k}=1) may be placed on resistance that had already been acquired by the patient before the first recorded treatment. Such resistance may be acquired due to past treatments missing from the data before t=1, or due to an initial infection by a drug resistant strain. In cART, the therapy is expected to succeed if the virus is sensitive to at least one of the compounds in it, for at least one k, m_{t,k}=0. To account for deviations from this model, and since medical records are prone to errors, the observed outcome may be modeled as a noisy version of this expected outcome. Let Oₜ^{E} be the expected therapy outcome, and let Oₜ be the therapy outcome observed in the EHR data, then

O^{E}=∨_{{k:d}_{_{t,k}}₌₁}¬m_{t,k}

Pr(Oₜ=Oₜ^{E}|\vec{mₜ},\vec{dₜ})=1−p^{N}. (3)

At any given time t, the observed treatment outcome, Oₜ, depends only on a small number of variables m_{t,k} (typically 2-3) associated with the drugs in the cART. The generative process may then be described as follows:

1. For all drug compounds k=1, . . . , K
(a) draw mutation probability pₖ^{M}˜Beta (β)
(b) draw prior resistance probability pₖ^{R}^{₀}˜Beta (γ)

2. draw outcome noise probability pₖ^{N}˜Beta (η)

3. For all patients i=1, . . . , N
(a) for t=1, . . . , T
   i. for all drugs k=1, . . . , K
      A. if t=1, draw R_{i,1,k}˜pₖ^{R}^{₀}, else draw R_{i,t,k}˜Pr(R_{i,t,k}|R_{i,t-1,k},O_{i,t-1},m_{i,t-1,k})
      B. draw m_{i,t,k} conditioned on R_{i,t,k},d_{i,t,k}
   ii. draw treatment outcome Oₜ conditioned on \vec{m}_{i,t},\vec{d}_{i,t},
      where N is the number of patients, K is the number of available drug compounds for treating the disease, and β, γ, and η are Beta priors treated as hyper-parameters of the model. This model is depicted in FIG. 4.

In some embodiments, the Factorial HMM model of the present invention may comprise an advantageous Collapsed Gibbs Sampling algorithm. In collapsed Gibbs Sampling, the discrete variables are sampled while the continuous variables are integrated out. In the present case, the discrete variables need to be sampled are m and R, and a. The continuous variables integrated out are p^{M},p^{R}^{₀},p^{N}, and p^{α}. The Beta priors are assumed to be known and set ϵ=0.01. The model then iterates over all patients and their treatments t, and at each iteration sample \vec{Rₜ},\vec{m}ₜ,aₜ of a specific patient conditioned on all other variables (omitting again the patient specific subscripts for ease of notation). Due to the deterministic relations between \vec{Rₜ},\vec{m}ₜ,aₜ in some cases, (e.g. m_{t,k}=1 and R_{t,k}=0 and a_{t,k}=1), these 2K+1 parameters are sampled together as a block.

The factorization of the joint posterior distribution noted above may be used to sample them efficiently (in time linear, rather than exponential, in the number of prescribed drugs). \vec{Rₜ},\vec{m}ₜ,aₜ are sampled conditioned on all other variables, observations and hyper parameters. To simplify and shorten the equations below, only the variables on which \vec{Rₜ},\vec{m}ₜ,aₜ depend are written explicitly, while all others are omitted. The hyper parameters and observed prescriptions are also omitted.

Pr(\vec{R}ₜ,\vec{m}ₜ,aₜ|\vec{R}ₜ₋₁,\vec{R}ₜ₊₁,Oₜ₋₁,Oₜ)

=Pr(\vec{R}ₜ|\vec{m}ₜ,aₜ,\vec{R}ₜ₋₁,\vec{R}ₜ₊₁,Oₜ₋₁)

Pr(\vec{m}ₜ|\vec{R}ₜ₋₁,\vec{R}ₜ₊₁,Oₜ₋₁,Oₜ)

Pr(aₜ|Oₜ₋₁,Oₜ,\vec{R}ₜ₋₁,\vec{R}ₜ₊₁) (4)

The factorization given by the equations above implies that aₜ is first sampled averaging over the 2K variables \vec{R}ₜ,\vec{a}ₜ, then \vec{m}ₜ is sampled conditioned on the already sampled aₜ and averages over the \vec{R}ₜ, and finally \vec{R}ₜ is sampled conditioned on aₜ,\vec{m}ₜ.

Thus, \vec{R}ₜ is first sampled, where the individual elements R_{t,k} of \vec{R}ₜ are conditionally independent given aₜ,mₜ,Rₜ₊₁,Rₜ₋₁,Oₜ, which may be then sampled individually:

Pr(R_{t,k}|R_{t-1,k},R_{t+1,k},mₜ,aₜ,Oₜ₋₁,Oₜ)

∝Pr(Rₜ₊₁|R_{t,k},Oₜ,m_{t,k})

Pr(m_{t,k}|R_{t,k},aₜ)

Pr(R_{t,k}|R_{t-1,k},Oₜ₋₁). (5)

For sampling \vec{m}ₜ, the factorization Pr(\vec{m}ₜ| . . . )=ΠₖPr(m_{t,k}|m_{t,k′}<k) may be used to individually sample \vec{m}ₜ conditioned on m_{t,k′} for all k′<k, and averaged over m_{t,k′} for all k′>k, as described above:

$$\begin{matrix}Pr ( {m}_{t , k} \vert {\vec{R}}_{t - 1} , {\vec{R}}_{t + 1} , {a}_{t} , {o}_{t} , {o}_{t - 1} , {m}_{t , k ′ < k} ) & ( 6 ) \\ ∝ {\sum }_{{R}_{t , k}} Pr ( {R}_{t + 1 , k} \vert {m}_{t} , {O}_{t} , {R}_{t , k} ) & \\ Pr ( {O}_{t} \vert {m}_{t , k ′ \leq k} , {\hat{R}}_{t - 1 , {k}^{′} > k} , {\hat{R}}_{t + 1 , {k}^{′} > k} ) & \\ Pr ( {m}_{t , k} \vert {a}_{t} , {R}_{t , k} ) Pr ( {R}_{t , k} \vert {R}_{t - 1 , k} , {O}_{t - 1} ) . & \end{matrix}$$

Computing Pr(Oₜ|m_{t,k},\vec{R}ₜ₋₁,\vec{R}ₜ₊₁,m′_{t,k}<k) requires averaging over all configurations of m_{t,k′>k} (an exponentially large number). Accordingly, the equation below shows how to compute this in linear time:

$$\begin{matrix}Pr ( {O}_{t} \vert {m}_{t , k ′ \leq k} , {o}_{t - 1} , {\vec{R}}_{t - 1 , {k}^{′} > k} , {\vec{R}}_{t + 1 {k}^{′} > k} ) & ( 7 ) \\ ∝ {\sum }_{o - 0 , 1} {\sum }_{m ∈ {M}_{o}}^{ } [ Pr ( {O}_{t} \vert {O}^{E} ( {\vec{m}}_{t} = 0 ) ) Pr ( {\vec{m}}_{t , {k}^{′} > k} \vert {a}_{t} , {R}_{t - 1 , {k}^{′} > k} , {O}_{t - 1} ) Pr ( {R}_{t + 1 , {k}^{′} > k} \vert {O}_{t} , {m}_{t , {k}^{′}} ) ) ] , & \end{matrix}$$

where M₁={m: O^{E}(m)=1} and M₀={m: O^{E}(m)=0} are the sets of all configurations of \vec{m} that yield the specific expected outcome O^{E}. The sum over M₀ includes the single term where all mₖ=1 (for drugs prescribed at time t). The other factors from equation 6 may be computed using equations 2 and 11. The other sum, over M₁ includes all other configurations. It is computed in time linear in the number of drugs in the therapy using the factorization:

$$\begin{matrix} Pr ( {\vec{m}}_{t} \vert {\vec{R}}_{t - 1} , {a}_{t} ) Pr ( {\vec{R}}_{t + 1} \vert {O}_{t} , {\vec{m}}_{t} , {a}_{t} ) & ( 8 ) \\ {\prod }_{k} [ {\sum }_{{R}_{k}} P r ( {m}_{t , k} \vert {R}_{t , k} , {a}_{t} ) Pr ( {R}_{t + 1} \vert {O}_{t} , {m}_{t , {k}^{′}} {R}_{t , {k}^{′}} ) Pr ( {R}_{t , k} \vert {R}_{t - 1 , k} , {0}_{t - 1} ) ] & \end{matrix}$$

Similarly, aₜ is sampled while averaging over \vec{m}ₜ,\vec{R}ₜ in linear time:

Pr(aₜ|Oₜ₋₁,Oₜ,\vec{R}ₜ₋₁,\vec{R}ₜ₊₁) (9)

∝Pr(aₜ)Pr(Oₜ|aₜ,\vec{R}ₜ₋₁,\vec{R}ₜ₊₁) (10)
where Pr(Oₜ|aₜ,\vec{R}ₜ₋₁,\vec{R}ₜ₊₁) is computed using equation 7 above setting k′=0 (i.e., averaging over all mₖ;Rₖ).

The other probabilities required for sampling according to equations 5-7 are computed from counts of sampled variables:

$$\begin{matrix}P r ( {m}_{t , k} = m \vert {R}_{t , k} = 0 , {a}_{t} = 1 ) = \frac{{c}_{k , m}^{M} + β}{{\sum }_{{m}^{′}} {c}_{k , {m}^{′}}^{M} + 2 β} & ( 11 ) \\ Pr ( {O}_{t} \vert {\vec{m}}_{t} ) = \frac{{c}_{{o}_{t} = {o}_{t}^{E} ( m )} + η}{{\sum }_{n} {c}_{n} + 2 η} & ( 12 ) \\ P r ( {R}_{1 , k} = r ) = \frac{{c}_{k r}^{{R}_{0}} + γ}{{\sum }_{{r}^{′}} {c}_{k , {r}^{′}}^{{R}_{0}} + 2 γ} & ( 13 ) \\ P r ( {a}_{i , t} = a ) = \frac{{c}_{i , a}^{A} + α}{{\sum }_{{a}^{′}} {c}_{i , {a}^{′}}^{A} + 2 α} & ( 14 )\end{matrix}$$

where C_{k,m}^{M},C_{k,r}^{R}^{₀},C_{i,a}^{A} are the counts of the sampled variables m_{.,k},n_{i,t},R_{0,k},aᵢ respectively, excluding \vec{m}_{i,t},\vec{R}_{0,i,k},a_{i,t}, and Cₙ counts the number of times Oₜ is equal or different from Oₜ^{E} (computed from the sampled m's).

To summarize, despite the coupling of the chains through patient adherence and therapy outcome, a sampling algorithm was derived that is linear in the number of variables to be sampled. This is in contrast to sampling in a general Factorial HMM.

Although the above descriptions highlight use of the invention through an example regarding HIV, it will be appreciated by those of ordinary skill in the art that the invention lends itself to many different variations not specifically illustrated herein. In practice, the invention described herein may be used to provide patient adherence estimation for any long-term pathogen or condition capable of acquiring resistances to treatments.

With reference back to the flowchart in FIG. 3, at a step 304, data analysis module 202b analyzes the patient data received in step 302 and calculates probabilities of patient non-adherence and/or pathogen resistance in patient. For example, data analysis module 202b may infer that the patient has not adhered to the prescribed medication regimen, e.g., from variations in the viral load levels and the fact that measured decreases in viral load levels are not temporally aligned with the prescribed medication intervals. At the same time, because viral load levels show a decrease at certain points, data analysis module 202b may further infer indicating a smaller likelihood that drug resistance has been built up by the pathogen. In another example, data analysis module 202b may infer appropriate adherence by a patient to the medication regimen based on, e.g., a sustained decrease in viral load levels, which also indicates lack or resistance build up. In other cases, data analysis module 202b may infer from a consistent return of viral load to initial levels may also that a virus resistance has been built up.

At a step 306, reporting module 202c may be configured for reporting the results of the analysis to the user, e.g., through user interface 210.

The present invention may be a system, a method, and/or a computer program product. The computer program product may include a computer readable storage medium (or media) having computer readable program instructions thereon for causing a processor to carry out aspects of the present invention.

The computer readable storage medium can be a tangible device that can retain and store instructions for use by an instruction execution device. The computer readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire. Rather, the computer readable storage medium is a non-transient (i.e., not-volatile) medium.

Computer readable program instructions described herein can be downloaded to respective computing/processing devices from a computer readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present invention may be assembler instructions, instruction-set-architecture (ISA) instructions, machine instructions, machine dependent instructions, microcode, firmware instructions, state-setting data, or either source code or object code written in any combination of one or more programming languages, including an object oriented programming language such as Java, Smalltalk, C++ or the like, and conventional procedural programming languages, such as the “C” programming language or similar programming languages. The computer readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In some embodiments, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry, in order to perform aspects of the present invention.

Aspects of the present invention are described herein with reference to flowchart illustrations and/or block diagrams of methods, apparatus (systems), and computer program products according to embodiments of the invention. It will be understood that each block of the flowchart illustrations and/or block diagrams, and combinations of blocks in the flowchart illustrations and/or block diagrams, can be implemented by computer readable program instructions.

These computer readable program instructions may be provided to a processor of a general-purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

The flowchart and block diagrams in the Figures illustrate the architecture, functionality, and operation of possible implementations of systems, methods, and computer program products according to various embodiments of the present invention. In this regard, each block in the flowchart or block diagrams may represent a module, segment, or portion of instructions, which comprises one or more executable instructions for implementing the specified logical function(s). In some alternative implementations, the functions noted in the block may occur out of the order noted in the figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

The descriptions of the various embodiments of the present invention have been presented for purposes of illustration, but are not intended to be exhaustive or limited to the embodiments disclosed. Many modifications and variations will be apparent to those of ordinary skill in the art without departing from the scope and spirit of the described embodiments. The terminology used herein was chosen to best explain the principles of the embodiments, the practical application or technical improvement over technologies found in the marketplace, or to enable others of ordinary skill in the art to understand the embodiments disclosed herein.

What is claimed is:

1. A system comprising:
at least one hardware processor; and
a non-transitory computer-readable storage medium having stored thereon program instructions, the program instructions executable by the at least one hardware processor to:
receive a dataset comprising:
(i) a treatment plan for a subject, the treatment plan comprising a plurality of treatment events scheduled at specified intervals, and
(ii) clinical outcomes of said subjects observed during said treatment plan, and
automatically analyze said dataset to determine adherence by said subject to said treatment plan.

2. The system of claim 1, wherein the analyzing comprises applying one or more stochastic models to said dataset.

3. The system of claim 2, wherein the one or more models includes a Factorial Hidden Markov Model.

4. The system of claim 1, wherein said analyzing is performed, at least in part, via one or more approximate learning methods.

5. The system of claim 4, wherein the one or more approximate learning methods include Collapsed Gibbs Sampling.

6. The system of claim 1, wherein said analyzing further determines effectiveness of said treatment plan.

7. A method comprising:
receiving a dataset comprising:
(i) a treatment plan for a subject, the treatment plan comprising a plurality of treatment events scheduled at specified intervals, and
(ii) clinical outcomes of said subjects observed during said treatment plan; and
automatically analyzing said dataset to determine adherence by said subject to said treatment plan.

8. The method of claim 7, wherein the analyzing comprises applying one or more stochastic models to said dataset.

9. The method of claim 8, wherein the one or more models includes a Factorial Hidden Markov Model.

10. The method of claim 7, wherein said analyzing is performed, at least in part, via one or more approximate learning methods.

11. The method of claim 10, wherein the one or more approximate learning methods include Collapsed Gibbs Sampling.

12. The method of claim 7, wherein said analyzing further determines effectiveness of said treatment plan.

13. A computer program product comprising a non-transitory computer-readable storage medium having program code embodied therewith, the program code executable by at least one hardware processor to:
receive a dataset comprising:
(i) a treatment plan for a subject, the treatment plan comprising a plurality of treatment events scheduled at specified intervals, and
(ii) clinical outcomes of said subjects observed during said treatment plan; and
automatically analyze said dataset to determine adherence by said subject to said treatment plan.

14. The computer program product of claim 13, wherein the analyzing comprises applying one or more stochastic models to said dataset.

15. The computer program product of claim 14, wherein the one or more models includes a Factorial Hidden Markov Model.

16. The computer program product of claim 13, wherein said analyzing is performed, at least in part, via one or more approximate learning methods.

17. The computer program product of claim 16, wherein the one or more approximate learning methods include Collapsed Gibbs Sampling.

18. The computer program product of claim 13, wherein said analyzing further determines effectiveness of said treatment plan.
